(19) United States
(12) Patent Application Publication
(10) Pub. No.: US 2010/0076052 A1
(43) Pub. Date: Mar. 25, 2010

(54) PERINDOPRIL FORMULATIONS

(76) Inventors: Janardhanan Anand Subramony, Lexington, MA (US); Jyotsna Rath, Bilaspur (IN); Prangya Paramita Pati, Sambalpur (IN)

Correspondence Address: DR. REDDY''S LABORATORIES, INC., 200 SOMERSET CORPORATE BLVD, SEVENTH FLOOR, BRIDGEWATER, NJ, 08807-2862, US

(21) Appl. No.: 12/565,424

(22) Filed: Sep. 23, 2009

Related U.S. Application Data
(60) Provisional application No. 61/141,286, filed on Dec. 30, 2008.

(30) Foreign Application Priority Data
Sep. 24, 2008 (IN) 2317/CHE/2008

(51) Int. Cl. A61K 31/40

(52) U.S. Cl. 514/419

(57) ABSTRACT

Pharmaceutical formulations comprising perindopril or its salts, isomers, enantiomers, polymorphs, metabolites, solvates, hydrates, and mixtures thereof, and at least one surface stabilizer. Also disclosed are methods of stabilizing perindopril in the formulations, and polyoxyethylene-polyoxypropylene block copolymers as surface stabilizers.

### INTRODUCTION

Aspects of the present invention relate to pharmaceutical formulations comprising perindopril or its salts, isomers, enantiomers, polymorphs, metabolites, solvates, hydrates, and mixtures thereof. Further aspects of the present invention relate to stable pharmaceutical formulations comprising perindopril, and processes to prepare these formulations.

The drug having the adopted name “perindopril erbumine” has a chemical name (2S,3αS,7αS)-1-[(S)—N—[(S)-1-carboxybutyl]alanyl]hexahydro-2-indolinecarboxylic acid, 1-ethyl ester, compound with t-butylamine (1:1). Its molecular formula is C₁₉H₃₂N₂O₅C₄H₁₁N and the structural formula is (1).

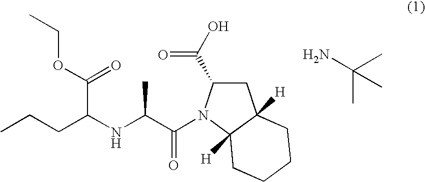

Perindopril erbumine is a white, crystalline powder with a molecular weight of 441.61. It is freely soluble in water (60% w/w), alcohol and chloroform. Perindopril (molecular weight 368.47) is the free acid form of perindopril erbumine, is a pro-drug, and is metabolized in vivo by hydrolysis of the ester group to form perindoprilat, a biologically active metabolite.

Perindopril erbumine is available in the U.S. in products sold by Solvay Pharmaceuticals as ACEON® tablets with 2 mg, 4 mg and 8 mg strengths for oral administration. In addition to perindopril erbumine, each tablet contains the following inactive ingredients: colloidal silica (hydrophobic), lactose, magnesium stearate and microcrystalline cellulose. The 4 and 8 mg tablets also contain iron oxide.

Perindopril erbumine is also available in the European market in COVERSYL® tablets with 2 mg, 4 mg and 8 mg strengths for oral administration. In addition to perindopril erbumine, each tablet contains the following inactive ingredients: microcrystalline cellulose, lactose monohydrate, hydrophobic colloidal silica, magnesium stearate and, for 4 mg and 8 mg tablets, aluminium complexes of chlorophyllins lake.

Perindopril and perindoprilat inhibit the angiotensin-converting enzyme (ACE). It is indicated for use in patients with stable coronary artery disease to reduce the risk of cardiovascular mortality or non-fatal myocardial infarction.

Perindopril is disclosed in U.S. Pat. Nos. 4,508,729 and 5,162,362. International Application Publication Nos. WO 2004/113293, WO 2004/046172, WO 2005/068425, WO 2005/037788, and WO 2007/092758. European Patent Nos. 1296947, 1294689, and 1296948 disclose various polymorphic forms of perindopril or its salts.

International Application Publication Nos. WO 2005/007130, WO 2003/075842, WO 2002/45693, WO 2003/059388, WO 2005/002548, WO 2005/041940, WO 2005/082420, WO 2005/007130, WO 2005/094793, WO 2005/084670, and WO 2003/075842 disclose pharmaceutical formulations containing ACE inhibitors.

Generally, it is known that drugs or drug products, when exposed to different environmental conditions, are prone to different reactions, which may cause drug to degrade and generate impurities. In addition to this, during manufacturing process, the drug or drug product may also be subjected to attrition/pressure such as during mixing, granulation, drying, milling, etc. Due to this, the drug may lose its nature and may be converted into other polymorphic forms. This poses challenges to the formulation scientist to choose appropriate excipients to stabilize the drug in the formulation.

From the literature, it is known that ACE inhibitors are drugs that are susceptible to degradation upon contact with many of the excipients commonly used in pharmaceutical products, or interconversion into other polymorphic forms under environmental influences such as elevated temperature and high relative humidity.

It is believed that one or more of these types of degradation products may result in decreased drug effectiveness in pharmaceutical formulations comprising perindopril or its salts. In addition, the degradation products may result in decreased drug effectiveness in such pharmaceutical formulations. It is known that when a drug substance contains even a trace amount of impurities above the acceptable limits, a possibility of an adverse influence on diagnosis and therapeutic treatment exists.

Different methods of stabilizing ACE inhibitors in pharmaceutical formulations are known. For example, pharmaceutical formulations comprising ACE inhibitors can be stabilized by the presence of alkali or alkaline earth metal salts, magnesium oxide, hydrochloric acid donors, ascorbic acid etc. Hence, the design of pharmaceutical formulations of perindopril is a definitive challenge to a formulation scientist.

Although each of the above represents an attempt to overcome the instability problems associated with ACE inhibitor-containing formulations, there still exists a need for perindopril-containing formulations exhibiting improved stability.

### SUMMARY

Aspects of the present invention relate to pharmaceutical formulations comprising perindopril or its salts, isomers, racemates, enantiomers, hydrates, solvates, metabolites, and polymorphs, and processes to prepare the formulations.

Further aspects of the present invention relate to stable pharmaceutical formulations comprising perindopril or its salts.

In embodiments, the present invention includes stable formulations of perindopril or its salts, wherein perindopril or its salts retains its physical form in a formulation.

In embodiments, the invention includes stable pharmaceutical formulations comprising perindopril or its salts and at least one surface stabilizer.

In embodiments, the invention includes stable pharmaceutical formulations comprising perindopril or its salts, wherein a surface stabilizer comprises a surfactant.

In embodiments, the invention includes stable pharmaceutical formulations comprising perindopril or its salts, wherein a surface stabilizer comprises a nonionic surfactant.

In embodiments, the invention includes stable pharmaceutical formulations comprising perindorpil or its salts, wherein a surface stabilizer comprises polyoxyethylene-polyoxypropylene block copolymers, such as poloxamers.

Further aspects of the invention relate to processes to prepare pharmaceutical formulations comprising perindopril or its salts.

In an aspect, the invention includes methods of using the formulations of the present invention to treat cardiovascular disorders.

### BRIEF DESCRIPTION OF THE DRAWINGS

FIG. 1 shows comparative powder X-ray diffraction (XRD) patterns for the formulation prepared according to Example 1, wherein: pattern A represents the starting perindopril erbumine; pattern P represents a similarly prepared placebo formulation, omitting the drug; pattern B represents the formulation as initially prepared; and pattern C represents the formulation after storage at 25° C. and 60% relative humidity (RH) for 1 month.

FIG. 2 shows comparative XRD patterns for the formulation prepared according to Example 1, wherein: pattern A represents the starting perindopril erbumine; pattern P represents a similarly prepared placebo formulation, omitting the drug; pattern B represents the formulation after storage at 30° C. and 65% relative humidity for 7 days; and pattern C represents the formulation after storage at 30° C. and 65% RH for 15 days.

FIG. 3 shows comparative XRD patterns for the formulation prepared according to Example 2, wherein: pattern A represents the starting perindopril erbumine; pattern P represents a similarly prepared placebo formulation, omitting the drug; pattern B represents the formulation as initially prepared; and pattern C represents the formulation after storage at 25° C. and 60% RH for 1 month.

FIG. 4 shows comparative XRD patterns for the formulation prepared according to Example 4, wherein: pattern A represents the starting perindopril erbumine; pattern P represents a similarly prepared placebo formulation, omitting the drug; pattern B represents the formulation as initially prepared; and pattern C represents the formulation after storage at 40° C. and 75% RH for 15 days.

For all of the drawings, the vertical axis is intensity units and the horizontal axis is the 2θ angle, in degrees.

### DETAILED DESCRIPTION

Aspects of the present invention relate to pharmaceutical formulations comprising perindopril or its salts, isomers, racemates, enantiomers, hydrates, solvates, metabolites, and polymorphs, and processes to prepare the formulations.

Further aspects of the present invention relate to stable pharmaceutical formulations comprising perindopril or its salts.

In embodiments, the invention relates to stable formulations comprising perindopril or its salts together with surface stabilizers, wherein the perindopril or salt retains its physical stable form during storage for commercially relevant times under conditions such as room temperature (25° C. and 60% RH), intermediate conditions (30° C. and 65% RH), and accelerated stability testing conditions (40° C. and 75% RH).

There are a number of pharmaceutical formulations, which suffer from instability problems due to the fact that the active component is susceptible to certain types of degradation, thereby rendering them unsuitable from a commercial standpoint. For example, several angiotensin-converting enzyme (ACE) inhibitor-containing formulations suffer from this drawback since certain ACE inhibitors degrade readily in pharmaceutical dosage forms. Accordingly, in view of their usefulness in treating hypertension, a number of research endeavours have been directed to overcoming the instability problems associated with ACE inhibitor-containing formulations.

Perindopril, being an ACE inhibitor, is observed to be unstable both physically and chemically, thus leading to degradation in dosage forms to form diketopiperazones (the internal cyclization products), diacids (the ester hydrolysis products), etc.

Suitable physiologically acceptable salts of perindopril include salts formed with acids such as the following: hydrochloric acid, hydrobromic acid, phosphoric acid, sulfuric acid, methylsulfuric acid, amidosulfonic acid, nitric acid, formic acid, acetic acid, propionic acid, succinic acid, tartaric acid, lactic acid, malonic acid, fumaric acid, oxalic acid, citric acid, malic acid, mucic acid, benzoic acid, salicylic acid, aceturic acid, embonic acid, naphthalene-1,5-disulfonic acid, ascorbic acid, phenylacetic acid, p-aminosalicylic acid, hydroxyethanesulfonic acid, benzenesulfonic acid, and with synthetic resins containing acid groups. Also, salts may be formed with bases such as t-butylamine and arginine.

The term “pharmaceutical formulation” as used herein refers to dosage forms comprising perindopril and one or more excipients, wherein dosage forms may be solid oral dosage forms such as tablets, capsules, pills, granules, or sachets.

The term “stability” for purposes of the present invention relates to physical stability, chemical stability, and thermodynamic stability.

The term “surface stabilizer” as used in the present invention refers to a surface active agent, which stabilizes perindopril or its salts both physically and chemically. These include surfactants, which may be ionic or nonionic surfactants.

ACE inhibitors, upon contact with some of the commonly used pharmaceutical excipients, undergo degradation at accelerated rates due to any of:
i) Cyclization via internal nucleophilic attack to form substituted diketopiperazines.
ii) Hydrolysis of a side chain ester group.
iii) Oxidation to form products having unwanted coloration.

These drugs are therefore not sufficiently stable to enable a long product shelf life. It is thus generally difficult to select excipients that enable preparation of dosage forms with adequate stability.

Following are some of the impurities that may result from the degradation of perindopril in a pharmaceutical formulation.

1) Impurity A: (2S,3aS,7aS)-octahydro-1H-indole-2-carboxylic acid, represented by structural Formula II.

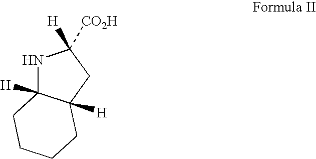

2) Impurity B: (2S,3aS,7aS)-1-[(2S)-2-[[(1S)-1-carboxybutyl]amino]propanoyl]octahydro-1H-indole-2-carboxylic acid, represented by structural Formula III, where R═H.

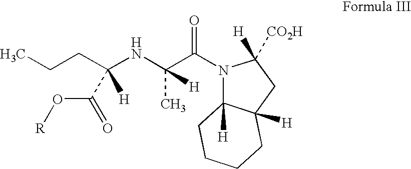

3) Impurity E: (2S,3aS,7aS)-1-[(2S)-2-[[(1S)-1-[(1-methylethoxy)carbonyl]butyl]amino]propanoyl]octahydro-1H-indole-2-carboxylic acid, represented by structural Formula III, where R═CH(CH₃)₂.

4) Impurity C: (2S)-2-[(3S,5aS,9aS,10aS)-3-methyl-1,4-dioxodecahydropyrazino[1,2-a]indol-2(1H)-yl]pentanoic acid, represented by structural Formula IV, where R═H.

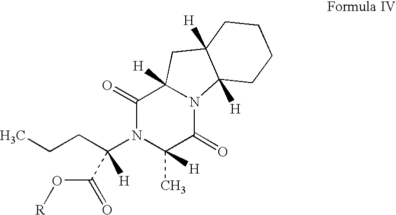

5) Impurity F: ethyl (2S)-2-[(3S,5aS,9aS,10aS)-3-methyl-1,4-dioxodecahydropyrazino[1,2-a]indol-2(1H)-yl]pentanoate, represented by structural Formula IV, where R═C₂H₅.

6) Impurity D: (2S)-2-[(3S,5aS,9aS,10aR)-3-methyl-1,4-dioxodecahydro pyrazino[1,2-a]indol-2(1H)-yl]pentanoic acid, represented by structural Formula V.

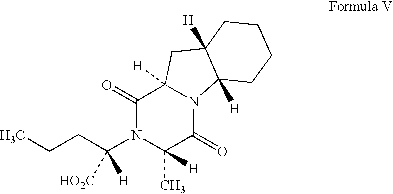

7) Impurity G: (2S,3aS,7aS)-1-[(2S)-2-[(5RS)-3-cyclohexyl-2,4-dioxo-5-propylimidazolidin-1-yl]propanoyl]octahydro-1H-indole-2-carboxylic acid, represented by structural Formula VI.

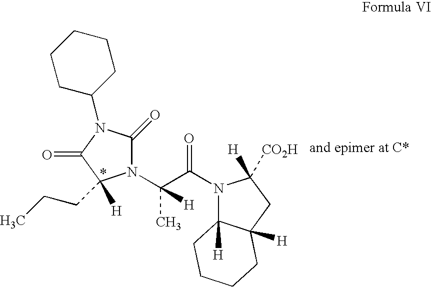

8) Impurity H: (2S,3aS,7aS)-1-[(2S)-2-[(5RS)-3-cyclohexyl-2-(cyclohexylimino)-4-oxo-5-propylimidazolidin-1-yl]propanoyl]octahydro-1H-indole-2-carboxylic acid, represented by structural Formula VII.

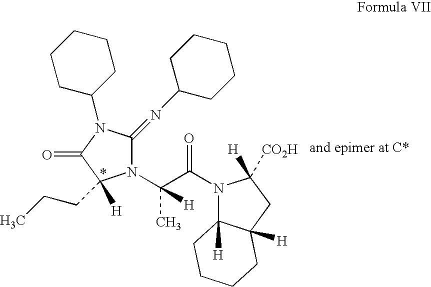

9) Impurity I: (2S,3aS,7aS)-1-[(2S)-2-[[(1R)-1-(ethoxycarbonyl)butyl]amino]propanoyl]octahydro-1H-indole-2-carboxylic acid, represented by structural Formula VIII.

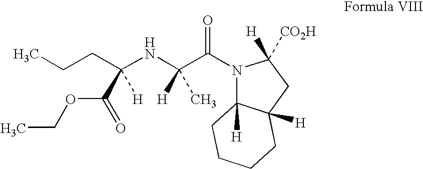

In embodiments, the invention relates to stable pharmaceutical formulations comprising perindopril or its salts.

In embodiments, the invention relates to stable pharmaceutical formulations comprising perindopril or its salts and at least one surface stabilizer.

Surface stabilizers can be ionic or nonionic in nature. Ionic surface stabilizers can be anionic, cationic, or zwitterionic.

Representative examples of useful surface stabilizers include, but are not limited to, sodium lauryl sulfate, dioctylsulfosuccinate, gelatin, casein, lecithin (phosphatides), dextran, gum acacia, cholesterol, tragacanth, hydroxypropyl methylcelluloses, hydroxypropylcelluloses, polyvinylpyrrolidones, polyoxyethylene alkyl ethers (e.g., macrogol ethers such as cetomacrogol 1000), polyoxyethylene castor oil derivatives, polyoxyethylene sorbitan fatty acid esters (such as the commercially available Tween™ products, e.g., Tween 20 and Tween 800 from ICI Specialty Chemicals), polyethylene glycols (e.g., Carbowax™ 3550 and 934 from Union Carbide), polyoxyethylene stearates, carboxymethylcellulose calcium, carboxymethyl cellulose sodium, methylcelluloses, hydroxyethylcelluloses, hydroxypropyl methylcellulose phthalates, magnesium aluminium silicate, triethanolamine, polyvinyl alcohols (PVA), poloxamers (e.g., Pluronic™ products F68 and F108Q, which are block copolymers of ethylene oxide and propylene oxide); poloxamines (e.g., Tetronic™ 908, also known as poloxamine 908, which is a tetrafunctional block copolymer derived from sequential addition of propylene oxide and ethylene oxide to ethylenediamine from BASF Wyandotte Corporation, Parsippany, N.J.), Tetronic™ 15080 (T-1508) (BASF Wyandotte Corporation), PEG-derivatized phospholipids, PEG-derivatized cholesterols, PEG-derivatized cholesterol derivatives, PEG-derivatized vitamin A, PEG-derivatized vitamin E, lysozyme, random copolymers of vinylpyrrolidone and vinyl acetate, and the like.

Examples of useful cationic surface stabilizers include, but are not limited to, polymers, biopolymers, polysaccharides, cellulosics, alginates, phospholipids, and nonpolymeric compounds, such as zwitterionic stabilizers, poly-n-methylpyridinium, anthryl pyridinium chloride, cationic phospholipids, chitosan, polylysine, polyvinylimidazole, polybrene, polymethylmethacrylate trimethylammoniumbromide bromide (PMMTMABr), hexyldesyltrimethyl ammonium bromide (HDMAB), polyvinylpyrrolidone-2-dimethylaminoethyl methacrylate dimethyl sulfate, 1,2-dipalmitoyl-sn-glycero-3-phosphoethanolamine-N-[amino(polyethylene glycol)2000] (sodium salt) (also known as DPPE-PEG (2000)-amine Na) (Avanti Polar Lipids, Alabaster, Ala.), poly(2-methacryloxyethyl trimethylammonium bromide) (Polysciences, Inc., Warrington, Pa.) (also known as S1001), poloxamines such as Tetronic 908, also known as Poloxamine 908m, which is a tetrafunctional block copolymer derived from sequential addition of propylene oxide and ethylene oxide to ethylenediamine (BASF Wyandotte Corporation, Parsippany, N.J.), lysozyme, long-chain polymers such as alginic acid, carrageenan (FMC Corp.), and POLYOX™ (Dow, Midland, Mich.).

Other useful cationic surface stabilizers include, but are not limited to, cationic lipids, sulfonium, phosphonium, and quarternary ammonium compounds, such as stearyltrimethylammonium chloride, benzyl-di(2-chloroethyl)ethylammonium bromide, coconut trimethyl ammonium chloride or bromide, coconut methyl dihydroxyethyl ammonium chloride or bromide, decyl triethyl ammonium chloride, decyl dimethyl hydroxyethyl ammonium chloride or bromide, C₂₋₅ dimethyl hydroxyethyl ammonium chloride or bromide, coconut dimethyl hydroxyethyl ammonium chloride or bromide, myristyl trimethyl ammonium methyl sulphate, lauryl dimethyl benzyl ammonium chloride or bromide, lauryl dimethyl (ethenoxy) 4 ammonium chloride or bromide, N-alkyl dimethylbenzyl ammonium chloride, N-alkyl dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts and dialkyl-dimethylammonium salts, lauryl trimethyl ammonium chloride, ethoxylated alkyamidoalkyldialkyl ammonium salt and/or an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl (C₁₂₋₁₄) dimethyl 1-naphthylmethyl ammonium chloride and dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride (ALIQUAT™ 336TM), tetrabutylammonium bromide, benzyl trimethylammonium bromide, choline esters (such as choline esters of fatty acids), benzalkonium chloride, stearalkonium chloride compounds (such as stearyltrimonium chloride and di-stearyldiammonium chloride), cetyl pyridinium bromide or chloride, halide salts of quaternized polyoxyethyl alkylamines, MIRAPOL™ and ALKAQUAT™ (Alkaril Chemical Company), alkyl pyridinium salts, amines, such as alkylamines, dialkylamines, alkanolamines, polyethylenepolyamines, N,N-dialkylaminoalkyl acrylates, and vinyl pyridine, amine salts, such as lauryl amine acetate, stearyl amine acetate, alkylpyridinium salt, and alkylimidazolium salt, and amine oxides, imide azolinium salts, protonated quaternary acrylamides, methylated quaternary polymers, such as poly[diallyl dimethylammonium chloride] and poly-[N-methyl vinylpyridinium chloride], and cationic guar.

Nonpolymeric cationic surface stabilizers include compounds such as benzalkonium chloride, carbonium compounds, phosphonium compounds, oxonium compounds, halonium compounds, cationic organometallic compounds, quarternary phosphorous compounds, pyridinium compounds, anilinium compounds, ammonium compounds, hydroxylammonium compounds, primary ammonium compounds, secondary ammonium compounds, tertiary ammonium compounds, and quarternary ammonium compounds.

In embodiments, the invention relates to stable pharmaceutical formulations comprising perindopril or a salt thereof and at least one surface stabilizer, wherein a surface stabilizer comprises a non-ionic surfactant.

In embodiments, the invention includes stable pharmaceutical formulations comprising perindopril or a salt thereof and at least one surface stabilizer, wherein a non-ionic surfactant comprises a polyoxyethylene-polyoxypropylene block copolymer, such as a poloxamer.

Poloxamers, also known by the trade name Pluronic™, are nonionic triblock copolymers composed of a central hydrophobic chain of polyoxypropylene, or poly(propylene oxide), flanked by two hydrophilic chains of polyoxyethylene, or poly(ethylene oxide). Because the lengths of the polymer blocks can be customized, many different poloxamers exist having slightly different properties. For the generic term “poloxamer”, these copolymers are commonly named with the letter “P” (for poloxamer) followed by three digits, the first two digits×100 give the approximate molecular mass of the polyoxypropylene core, and the last digit×10 gives the percentage polyoxyethylene content (e.g., P407=poloxamer with a polyoxypropylene molecular mass of 4,000 g/mole and a 70% polyoxyethylene content).

Different grades of poloxamers are commercially available such as poloxamer 407, poloxamer 401, poloxamer 237, poloxamer 338, poloxamer 331, poloxamer 231, poloxamine 908, poloxamine 1307, poloxamine 1107, and polyoxyethylene-polyoxybutylene block copolymer.

In embodiments, the present invention includes stable formulations comprising perindopril or its salts wherein perindopril or its salt retains its physical form in the formulations, i.e., formulations comprising perindopril of the present invention initially as well as after stability testing retain the characteristics of the perindopril or salt.

In embodiments, the invention relates to pharmaceutical formulations comprising perindopril or its salt, wherein a concentration of surface stabilizer or stabilizing amount of surface stabilizer is in the range of about 0.1% to about 75% or from about 0.5% to about 50%, by weight of the total formulation.

In embodiments, the invention includes weight ratios of perindorpil or its salts to surface stabilizer in the range of from about 1:0.01 to about 1:25, or from about 1:0.01 to about 1:20, or from about 1:0.1 to about 1:15.

In specific embodiments, weight ratios of perindopril erbumine to surface stabilizer are in the range of about 0.1 to about 5, or about 0.1 to about 2.5.

In embodiments, the invention includes pharmaceutical formulations comprising perindopril or its salts wherein a moisture content is not more than about 8% w/w.

In embodiments, the invention includes formulations comprising perindopril or its salts that, after exposure to accelerated stability testing conditions, e.g., 40° C. and 75% relative humidity (RH), substantially retain the initial XRD pattern for a commercially relevant time.

In embodiments, the invention includes formulations comprising perindopril or its salts that, after exposure to intermediate stability testing conditions, e.g., 30° C. and 65% relative humidity (RH), substantially retains the initial XRD pattern for a commercially relevant time.

In embodiments, the invention includes formulations comprising perindopril or its salts that, after exposure to room temperature stability testing conditions, e.g., 25° C. and 60% relative humidity (RH), substantially retains the initial XRD pattern for a commercially relevant time.

A commercially relevant time varies, such as about 15 days, 1 month, 6 months, 1 year, 2 years, etc., depending on the intended storage conditions. For products that are to be stored at room temperature, stability during a shelf life of one year or two years frequently is desired.

The formulations and pharmaceutical formulations of the present invention optionally include pharmaceutically acceptable excipients, in addition to surface stabilizers, such as but not limited to diluents or fillers, binders, disintegrants, glidants, lubricants, colorants, flavors, solvents, sweeteners, and film-forming polymers and other coating adjuvants.

Fillers or diluents include, but are not limited to, starches, lactose, mannitol, cellulose derivatives, confectioner's sugar and the like. Different grades of lactose include but are not limited to lactose monohydrate, lactose DT (direct tableting), and lactose anhydrous, Flowlac™ (available from Meggle Products), Pharmatose™ (available from DMV) and others. Different grades of starches include but are not limited to maize starch, potato starch, rice starch, wheat starch, pregelatinized starch (commercially available as PCS PC10 from Signet Chemical Corporation) and Starch 1500, Starch 1500 LM grade (low moisture content grade) from Colorcon, fully pregelatinized starch (commercially available as National 78-1551 from Essex Grain Products), and others. Cellulose compounds that can be used include crystalline celluloses and powdered celluloses. Examples of available crystalline cellulose products include, but are not limited to, CEOLUS™ KG801, Avicel™ PH101, PH102, PH301, PH302 and PH-F20, microcrystalline cellulose 114, and microcrystalline cellulose 112. Other useful diluents include, but are not limited to, carmellose, sugar alcohols such as mannitol, sorbitol and xylitol, calcium carbonate, magnesium carbonate, dibasic calcium phosphate, and tribasic calcium phosphate.

Binders include, but are not limited to, hydroxypropyl celluloses (e.g., Klucel™ LF), hydroxypropyl methylcelluloses or hypromelloses (e.g., Methocel™) polyvinylpyrrolidones or povidones (PVP-K25, PVP-K29, PVP-K30, and PVP-K90), Plasdone™ S 630 (copovidone), powdered acacia, gelatin, guar gum, carbomers (e.g. Carbopol™ products), methylcelluloses, polymethacrylates, and starches.

Disintegrants include, but are not limited to, carmellose calcium (Gotoku Yakuhin Co., Ltd.), carboxymethylstarch sodium (Matsutani Kagaku Co., Ltd., Kimura Sangyo Co., Ltd., etc.), croscarmellose sodium (FMC-Asahi Chemical Industry Co., Ltd.), crospovidones, examples of commercially available crospovidone products including but not limited to crosslinked povidone, Kollidon™ CL [manufactured by BASF (Germany)], Polyplasdone™ XL, XI-10, and INF-10 [manufactured by ISP Inc. (USA)], and low-substituted hydroxypropylcelluloses. Examples of low-substituted hydroxypropyl celluloses include, but are not limited to, the low-substituted hydroxypropylcelluloses LH11, LH21, LH31, LH22, LH32, LH20, LH30, LH32 and LH33 (all manufactured by Shin-Etsu Chemical Co., Ltd.). Other useful disintegrants include sodium starch glycolate, colloidal silicon dioxide, and starches.

Glidants or anti-sticking agents, include, but are not limited to, talc, silica derivatives, colloidal silicon dioxide, and the like and mixtures thereof.

Lubricants include, but are not limited to, stearic acid and stearic acid derivatives, for example, magnesium stearate, calcium stearate, zinc stearate, sucrose esters of fatty acids, polyethylene glycol, talc, sodium stearyl fumarate, zinc stearate, castor oils, and waxes.

Colourants include, but are not limited to, Food Yellow No. 5, Food Red No. 2, Food Blue No. 2, and the like, food lake colorants, and iron oxides.

Flavoring agents include, but are not limited to, those having a natural, synthetic, or semi-synthetic origin such as menthol, fruit flavors, citrus oils, peppermint oil, spearmint oil, and oil of wintergreen (methyl salicylate).

Sweeteners include, but are not limited to, natural sweeteners, for example sucrose, dextrose, fructose, invert sugar, mannitol, sorbitol and the like; and synthetic sweeteners, for example saccharin, aspartame, acesulfame potassium, cyclamates and the like. The amount of sweetener may vary depending on the sweetening strength of the particular sweetener used. Mixtures of any two or more sweeteners are useful in the invention.

Solvents that are useful in processing include, but are not limited to, water, ethanol, 1-butanol, 2-butanol, 3-methyl-1-butanol, 1-propanol, 2-propanol, 2-methyl-1-propanol, 1-pentanol, acetic acid, formic acid, heptane, anisole, acetone, ethyl acetate, butyl acetate, propyl acetate, isobutyl acetate, isopropyl acetate, methyl acetate, methylethyl ketone, methylisobutyl ketone, cumene, dimethylsulfoxide, pentanel, ethyl ether, t-butylmethyl ether, ethyl formate, and the like.

Film forming agents include, but are not limited to: soluble alkyl- or hydroalkylcellulose derivatives, for example, methylcelluloses, hydroxymethyl celluloses, hydroxyethyl celluloses, hydroxypropyl celluloses, hydroxymethyethyl celluloses, hydroxypropyl methylcelluloses, sodium carboxymethyl celluloses, etc.; acidic cellulose derivatives, for example, cellulose acetate phthalates, cellulose acetate trimellitates, and methyl hydroxypropylcellulose phthalates, polyvinyl acetate phthalates, etc.; insoluble cellulose derivatives, for example ethylcelluloses and the like; dextrins; starches and starch derivatives; polymers based on carbohydrates and derivatives thereof; natural gums, for example gum Arabic, xanthans, and alginates; polyacrylic acid; polyvinyl alcohols; polyvinyl acetate; polyvinylpyrrolidones; polymethacrylates and derivatives thereof (Eudragit™); chitosan and derivatives thereof; shellac and derivatives thereof; and waxes and fat substances.

Films may contain additional adjuvants for coating processes, for example plasticizers, polishing agents, colorants, pigments, antifoam agents, opacifiers, antisticking agents, and the like.

Plasticizers include, but are not limited to, castor oil, diacetylated monoglycerides, dibutyl sebacate, diethyl phthalate, glycerin, polyethylene glycol, propylene glycol, triacetin, triethyl citrate. Also, mixtures of plasticizers may be utilized. The type of plasticizer depends upon the type of coating agent.

An opacifier like titanium dioxide may also be present in an amount ranging from about 10% (w/w) to about 20% (w/w), based on the total weight of a coating. When coloured tablets are desired then the colour is normally applied in the coating. Consequently, colouring agents and pigments may be present in the film coating. Colouring agents include but are not limited to iron oxides, which can be red, yellow, black or blends thereof.

Anti-adhesives are frequently used in film coating processes to avoid sticking effects during film formation and drying. An example of a useful anti-adhesive for this purpose is talc. The anti-adhesive will be present in the film coating in an amount of about 5% (w/w) to 15% (w/w), based upon the total weight of the coating.

Polishing agents include, but are not limited to, polyethylene glycols of differing molecular weights or mixtures thereof, talc and surfactants (e.g. glycerol monostearate and poloxamers), fatty alcohols (e.g., stearyl alcohol, cetyl alcohol, lauryl alcohol and myristyl alcohol) and waxes (e.g., carnauba wax, candelilla wax and white wax).

In addition to the above coating ingredients, pre-mixed coating materials such as those sold as OPADRY (supplied by Colorcon) may be employed; these typically require only mixing with a liquid to form a sprayable coating formulation.

The formulations of the present invention may be formulated into solid oral dosage forms such as tablets, capsules, pills, granules, sachets, etc.

The formulations may be prepared by direct compression, wet granulation, or dry granulation processes.

In an aspect the invention includes processes for preparing formulations comprising perindopril or its salts, wherein an embodiment comprises:

1) Sifting excipients such as diluents, disintegrants, etc. and optionally active ingredient, through a sieve and mixing.

2) Preparing a granulating solution by dispersing or dissolving a suitable binder in a suitable solvent.

3) Granulating step 1) materials with granulating solution of step 3) and drying the granules.

4) Optionally, in place of steps 2) and 3), subjecting step 1) materials to roller compaction to form granules.

5) Sifting the dried granules of step 3) or granules of step 4) and extragranular excipients through a sieve, and blending.

6) Blending a lubricant with the blend of step 5).

7) Compressing the lubricated blend into tablets, or filling into capsules.

The equipment suitable for processing the formulation and formulations of the present invention include rapid mixer granulators, planetary mixers, mass mixers, ribbon mixers, fluid bed processors, mechanical sifters, blenders, roller compacter, compression machines, rotating bowls or coating pans, tray dryers, fluid bed dryers, rotary cone vacuum dryers, and the like, multimills, fluid energy mills, ball mills, colloid mills, roller mills, hammer mills, and the like, equipped with a suitable screen.

The formulation or dosage form prepared as above can be subjected to in vitro dissolution evaluation, such as according to Test 711 “Dissolution” in United States Pharmacopoeia 29, United States Pharmacopeial Convention, Inc., Rockville, Md., 2005, to determine the rate at which the drug substance is released from the dosage forms, and content of drug substance can be determined in solutions by techniques such as high performance liquid chromatography. Testing of the dosage forms for physical stability can involve powder X-ray diffraction (XRD), differential scanning calorimetry methods, solid NMR spectroscopy, infrared spectrophotometry, and other techniques.

The pharmaceutical dosage forms of the present invention are intended for oral administration to a patient in need thereof.

In embodiments, the invention includes use of packaging materials, for example containers and closures of high-density polyethylene (HDPE), low-density polyethylene (LDPE) and or polypropylene and/or glass, and blisters or strips composed of aluminum or high-density polypropylene, polyvinyl chloride, polyvinylidene dichloride, etc.

Certain specific aspects and embodiments of the invention will be further described in the following examples, which are provided solely for purposes of illustration and should not be construed to limit the scope of the invention in any manner.

### Example 1

### Pharmaceutical Formulation for Perindopril Erbumine 4 mg Tablets with 8% Poloxamer

| Ingredient | Grams |
|---|---|
| Perindropil erbumine | 0.896 |
| Poloxamer 407 | 1.6 |
| Microcrystalline cellulose (Avicel PH112) | 8.404 |
| Lactose monohydrate, impalpable | 8.8 |
| Colloidal silicon dioxide (Aerosil 200) | 0.1 |
| Magnesium stearate | 0.2 |

Manufacturing Process:

1) Poloxamer 407 is sifted through an ASTM #40 mesh sieve.

2) Perindopril erbumine is passed through an ASTM #40 mesh sieve.

3) Perindopril erbumine from step 2 is added to the material of step 1) and uniformly mixed for about 5 minutes.

4) Microcrystalline cellulose is sifted through an ASTM #40 mesh sieve and geometrically mixed with the mixture obtained in step 3).

5) Lactose is sifted through an ASTM #40 mesh sieve and is mixed geometrically with the mixture of step 4).

6) Colloidal silica is sifted through an ASTM #40 mesh sieve and is combined with the mixture obtained in step 5).

7) Magnesium stearate is sifted through an ASTM #60 mesh sieve, added to the mixture obtained in step 6), and blended for about 5 minutes.

8) The lubricated blend of step 7) is compressed into tablets having an average weight of 90 mg.

Tablets are packaged in amber coloured closed vials and stored at 25° C. and 60% RH for 1 month, and at 30° C. and 65% RH for 15 days. The stored samples are tested by XRD to determine change in the form of perindopril erbumine in the samples.

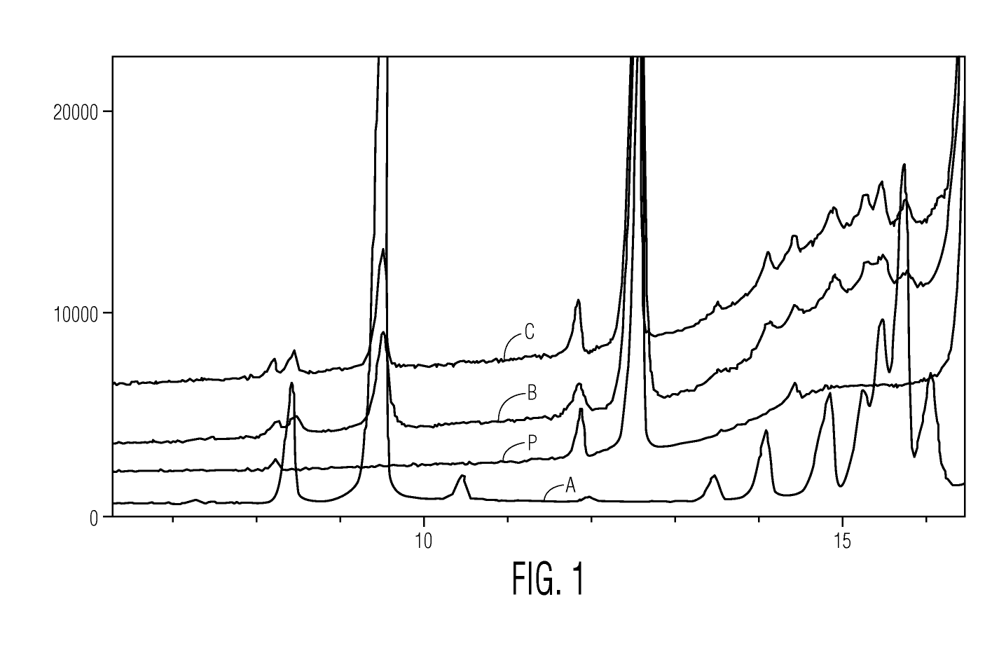

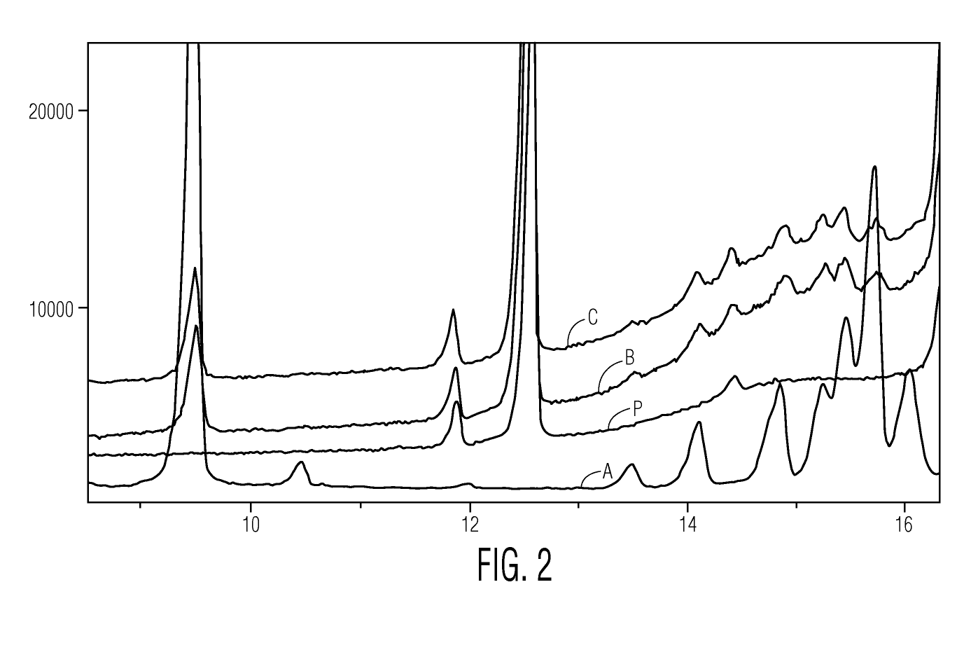

FIG. 1 is a comparison of powder X-ray diffraction (XRD) patterns, using copper Kα radiation, of: the perindopril erbumine ingredient (A); the formulation originally prepared (B); and the formulation after storage at 25° C. and 60% RH for 1 month (C). FIG. 2 is a comparison of the XRD patterns, using copper Kα radiation, of: the perindopril erbumine ingredient (A); the formulation prepared after storage for 7 days (B) and after storage for 15 days (C) at 30° C. and 65% RH. A “placebo” formulation is similarly prepared using the above ingredients, but omitting the perindorpil erbumine, and the XRD pattern of the placebo formulation is also shown (P). The XRD patterns of the formulation before storage matches that of the stored formulation, showing polymorphic stability.

### Example 2

### Pharmaceutical Formulation for Perindopril Erbumine 4 mg Tablets with 10% of Poloxamer

| Ingredient | Grams |
|---|---|
| Perindopril erbumine | 0.896 |
| Poloxamer 407 | 2 |
| Microcrystalline cellulose (Avicel PH112) | 8.204 |
| Lactose monohydrate impalpable | 8.6 |
| Colloidal silicon dioxide (Aerosil 200) | 0.1 |
| Magnesium stearate | 0.2 |

Manufacturing process: Similar to that of Example 1.

Tablets are packaged in amber coloured closed vials and stored at 25° C. and 60% RH for 1 month. The stored samples are tested by XRD to determine change in the form of perindopril erbumine in the samples.

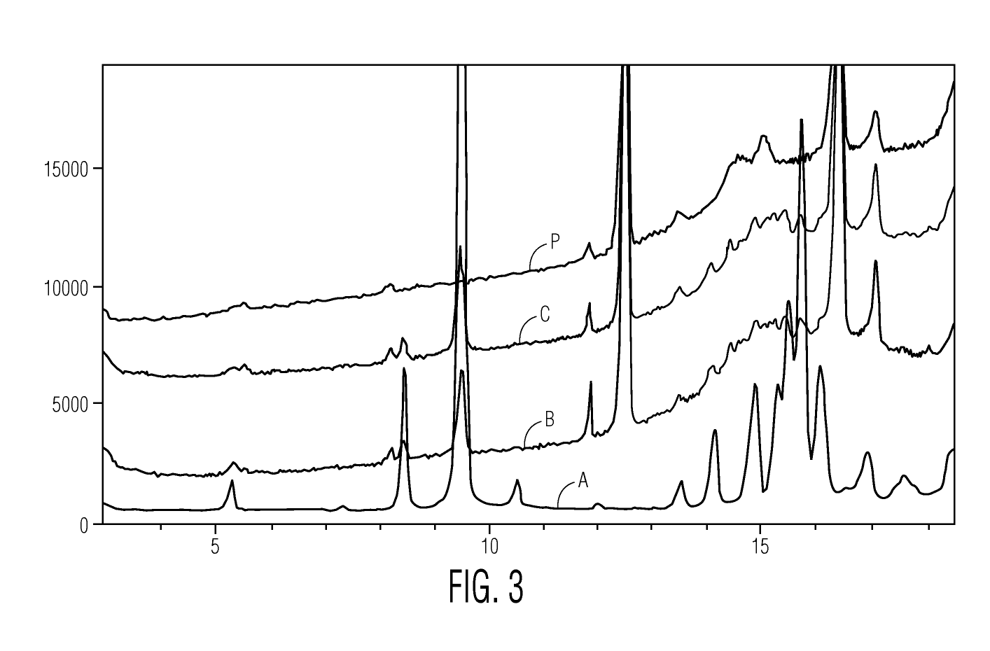

FIG. 3 is a comparison of XRD patterns, using copper Kα radiation, of: the perindopril erbumine ingredient (A); the formulation originally prepared (B); and the formulation after storage for 1 month (C). A “placebo” formulation is similarly prepared using the above ingredients, but omitting the perindopril erbumine, and the XRD pattern of the placebo formulation is also shown (P). The XRD pattern of the formulation before storage matches that of the stored formulation, showing polymorphic stability.

### Example 3

### Pharmaceutical Formulation for Perindopril Erbumine 4 mg Tablets with 15% of Poloxamer

| Ingredient | Grams |
|---|---|
| Perindopril erbumine | 0.896 |
| Poloxamer 407 | 3 |
| Microcrystalline cellulose (Avicel PH112) | 7.804 |
| Lactose monohydrate, impalpable | 8 |
| Colloidal silicon dioxide (Aerosil 200) | 0.1 |
| Magnesium stearate | 0.2 |

Manufacturing process: similar to that of Example 1.

### Example 4

### Pharmaceutical Formulation for Perindopril Erbumine 4 mg Tablets

| Ingredient | Grams |
|---|---|
| Perindopril erbumine | 0.4 |
| Poloxamer 407 | 4 |
| Microcrystalline cellulose (Avicel PH112) | 2.5 |
| Lactose monohydrate, impalpable | 2.95 |
| Colloidal silicon dioxide (Aerosil 200) | 0.05 |
| Magnesium stearate | 0.1 |

Manufacturing Process:

1) Perindopril erbumine and microcrystalline cellulose are uniformly mixed.

2) The step 1) mixture is uniformly mixed with lactose monohydrate.

3) The step 2) mixture is uniformly mixed with poloxamer.

4) The step 3) mixture is blended with colloidal silicon dioxide and then with magnesium stearate.

5) The lubricated blend of step 4) is compressed into tablets having a 250 mg average weight.

Tablets are tightly packaged in amber coloured closed vials and stored at 25° C. and 60% RH, and at 40° C. and 75% RH, for 15 days. The stored samples are tested by XRD to determine change in the form of perindopril erbumine in the samples.

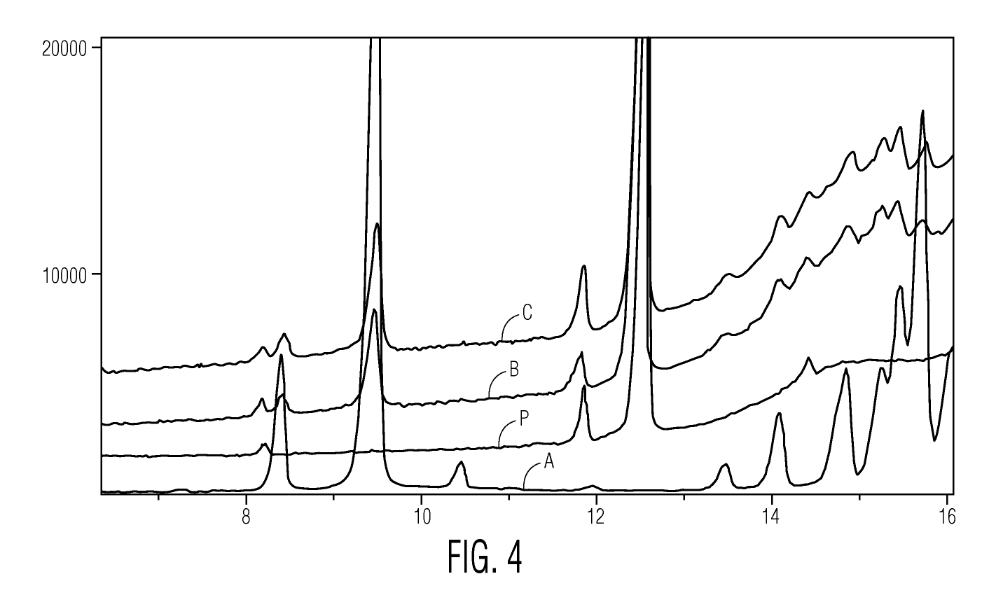

FIG. 4 is a comparison of the XRD patterns, using copper Kα radiation, of: the perindopril erbumine ingredient (A); the formulation originally prepared (B); and the formulation after storage at 40° C. and 75% RH(C). A “placebo” formulation is similarly prepared using the above ingredients, but omitting the perindorpil erbumine, and the XRD pattern of the placebo formulation is also shown (P). The XRD pattern of the formulation before storage matches that of the stored formulation, showing polymorphic stability.

### Example 5

### Formulation for Perindopril Erbumine 2 mg Tablets

| Ingredient | Grams |
|---|---|
| Perindropil erbumine | 0.242 |
| Poloxamer 407 | 0.108 |
| Microcrystalline cellulose (Avicel PH112) | 2.431 |
| Lactose monohydrate, impalpable | 2.535 |
| Colloidal silicon dioxide (Aerosil 200) | 0.027 |
| Magnesium stearate, | 0.054 |

Manufacturing process: similar to that of Example 1.

### Example 6

### Formulation for Perindopril Erbumine 4 mg Tablets

| Ingredient | Grams |
|---|---|
| Perindropil erbumine | 1.12 |
| Poloxamer 407 | 0.5 |
| Microcrystalline cellulose (Avicel PH112) | 11.255 |
| Lactose monohydrate, impalpable | 11.75 |
| Colloidal silicon dioxide (Aerosil 200) | 0.125 |
| Magnesium stearate | 0.25 |

Manufacturing process: similar to that of Example 1.

### Example 7

### Formulation for Perindopril Erbumine 8 mg Tablets

| Ingredient | Grams |
|---|---|
| Perindropil erbumine | 0.725 |
| Poloxamer 407 | 0.324 |
| Microcrystalline cellulose (Avicel PH112) | 7.293 |
| Lactose monohydrate, impalpable | 7.614 |
| Colloidal silicon dioxide (Aerosil 200) | 0.081 |
| Magnesium stearate | 0.162 |

Manufacturing process: similar to that of Example 1.

We claim:

1. A solid pharmaceutical formulation comprising perindopril or a salt thereof and a stabilizing amount of at least one surface stabilizer.

2. The pharmaceutical formulation of claim 1, wherein a weight ratio of perindopril or salt thereof to surface stabilizer is in the range of about 1:0.01 to about 1:25.

3. The pharmaceutical formulation of claim 1, wherein the concentration of surface stabilizer is in the range of about 0.1 to about 75% by weight.

4. The pharmaceutical formulation of claim 1, wherein a surface stabilizer is ionic or nonionic.

5. The pharmaceutical formulation of claim 1, wherein a surface stabilizer is a nonionic polymer.

6. The pharmaceutical formulation of claim 1, wherein a surface stabilizer is a polyoxyethylene-polyoxypropylene block copolymer.

7. The pharmaceutical formulation of claim 1, having a moisture content not more than about 8% by weight of the formulation.

8. The pharmaceutical formulation of claim 1, in the form of tablets.

9. The pharmaceutical formulation of claim 1, being packaged in a container that prevents moisture ingress.

10. A method of stabilizing a pharmaceutical formulation of claim 1, comprising geometrically mixing perindopril or a salt thereof and a surface stabilizer.

11. The pharmaceutical formulation of claim 1, wherein a powder X-ray diffraction pattern does not exhibit substantial change in the form of perindopril or a salt thereof, during storage in a closed container for 15 days at 40° C. and 75% relative humidity.

12. A solid pharmaceutical formulation comprising perindopril erbumine and a nonionic polymer surface stabilizer.

13. The pharmaceutical formulation of claim 12, wherein a nonionic polymer comprises a polyoxyethylene-polyoxypropylene block copolymer.

14. The pharmaceutical formulation of claim 12, wherein a nonionic polymer comprises poloxamer 407.

15. The pharmaceutical formulation of claim 12, wherein a nonionic polymer comprises poloxamer 407 and a weight ratio of perindopril erbumine to poloxamer 407 is between about 0.1 and about 5.

16. The pharmaceutical formulation of claim 12, in the form of tablets.

17. The pharmaceutical formulation of claim 12, having a moisture content not more than about 8% by weight of the formulation.

18. The pharmaceutical formulation of claim 12, wherein a powder X-ray diffraction pattern does not exhibit substantial change in the form of perindopril erbumine, during storage in a closed container for 15 days at 40° C. and 75% relative humidity.

19. A pharmaceutical formulation comprising perindopril erbumine and a polyoxyethylene-polyoxypropylene block copolymer, in the form of tablets.

20. The pharmaceutical formulation of claim 19, wherein a polyoxyethylene-polyoxypropylene block copolymer comprises poloxamer 407 and a weight ratio of perindopril erbumine to poloxamer 407 is between about 0.1 and about 5.
